(19) United States
(12) Patent Application Publication
(10) Pub. No.: US 2011/0152229 A1
(43) Pub. Date: Jun. 23, 2011

(54) BETULINIC ACID DERIVATIVES AS ANTI-HIV AGENTS

(75) Inventors: Chin Ho Chen, Chapel Hill, NC (US); Li Huang, Chapel Hill, NC (US); Kuo-Hsiung Lee, Chapel Hill, NC (US)

(73) Assignees: Duke University; The University of North Carolina at Chapel Hill

(21) Appl. No.: 11/943,956

(22) Filed: Nov. 21, 2007

Related U.S. Application Data
(60) Provisional application No. 60/866,935, filed on Nov. 22, 2006.

(51) Int. Cl. A61K 31/56; C07J 53/00; A61P 31/18

(52) U.S. Cl. 514/182; 552/510

(57) ABSTRACT

The present invention provides compounds of the general structure:

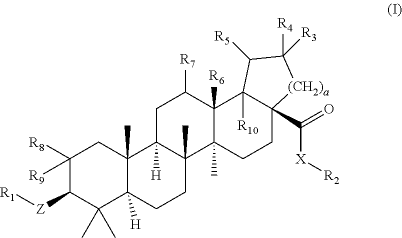

which are substituted at the 3 and 28 positions, along with pharmaceutical formulations containing the same and methods of treating viral infections employing the same.

### CROSS REFERENCE TO RELATED APPLICATIONS

This application claims priority to U.S. Provisional Application Ser. No. 60/866,935, filed Nov. 22, 2006, and the disclosure of which is incorporated by reference herein in its entirety.

### GOVERNMENT FUNDING

This invention was made, in-part, with United States government support under grant number AI 52022 and AI 50504 from the National Institutes of Health. The United States government has certain rights to this invention.

### FIELD OF THE INVENTION

The present invention concerns compounds, compositions and methods useful for the treatment of retroviral infections in human or animal subjects in need thereof.

### BACKGROUND OF THE INVENTION

Retroviruses are small, single stranded RNA viruses. Numerous species are susceptible to retroviral infection. While retroviral infection does not necessarily interfere with the normal life cycle of an infected cell or organism, retroviruses can be oncogenic, and retroviruses are responsible for diseases of the immune system in higher animals, including acquired immune deficiency syndrome (AIDS), caused by the human immunodeficiency virus (HIV).

Progress has been made in the development of drugs for HIV therapy. Among other things, U.S. Pat. No. 6,172,110 to Lee et al. describes acylated betulins for the treatment of HIV, U.S. Pat. No. 5,679,828 to Lee et al. describes betulinic acid derivatives for the treatment of HIV, U.S. Pat. No. 5,468,888 to Bouboutou et al. describes lupane derivatives for the treatment of HIV, and U.S. Pat. No. 7,026,305 to Chen et al. also describes betulinic acid derivatives for treatment of HIV. Specific examples of compounds that have been described for the treatment of HIV are the following:

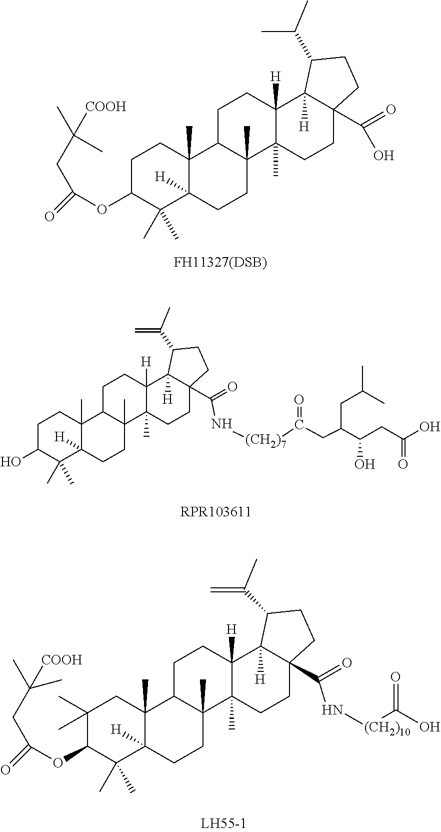

Nevertheless, many drugs exhibit severe toxicities, have side effects, require complicated dosing schedules, or—most problematically—can lead to the development of drug resistance thereto in the subject being treated. These problems are further exacerbated in countries where high cost multiple drug therapy is not readily affordable to the general population. Hence there remains a need for new compounds useful for treating HIV, including for the treatment of strains of HIV that are resistant to treatment with other known compounds. There particularly remains a need for new compounds that have novel modes of activity in treating HIV-1 infection.

### SUMMARY OF THE INVENTION

One aspect of the present invention is, accordingly, compounds useful for the treatment of a retroviral infection in need thereof.

A first aspect of the present invention is a compound according to Formula (I)

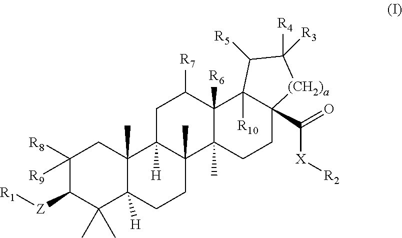

wherein:
a is 1 or 2;
Z is O, S, NH, or N-alkyl;
R₁ is a hydrogen,
or acyl carboxylic acid,
or R₁ is a substituent of the formula

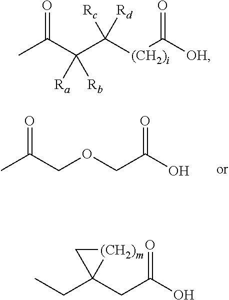

wherein Rₐ, R_{b}, R_{c} and R_{d} are the same or different and are each independently selected from the group consisting of hydrogen or lower alkyl, i is an integer from 0 to 3, and m is an integer from 1 to 4;
X is O, S, NH, or N-alkyl;
R₂ is a substituent of the formula:

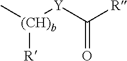

wherein:
   R′ and R″ are either hydrogen or alkyl radicals;
   Y is O, S, NH, N-alkyl, or heterocycle (e.g. piperazine);
   b is an integer from 0 to 16, or R₂ is benzotriazole;

R₃ and R₄ are either H or lower alkyl;

R₅ is H, lower alkyl or —CRᵢRᵢᵢRᵢᵢᵢ,

Rᵢ is a methyl radical or forms with Rₕ, a methylene radical or an oxo radical,

Rᵢᵢ is a hydroxyl, methyl or hydroxymethyl radical or a radical —CH₂ OR′ᵢᵢ, —CH₂SR′ᵢᵢ or —CH₂NHR′ᵢᵢ for which R′ᵢᵢ is alkyl, hydroxyalkyl, dihydroxyalkyl, acetamidoalkyl or acetyl, or Rᵢᵢ is an amino radical substituted with a hydroxyalkyl or carboxyhydroxyalkyl radical, or a dialkylamino radical, the alkyl parts of which can form, with the nitrogen atom to which they are joined, a 5- or 6-membered heterocycle optionally containing another hetero atom chosen from oxygen, sulphur or nitrogen and, optionally, N-alkyl;

Rᵢᵢᵢ is a hydrogen atom or forms, with Rᵢ, or Rᵢᵢ, a methylene radical or an oxo radical,

or R₅ form a bond with its immediately adjacent carbon atoms;

R₆, R₇ are the same or different and are either H or form bond with one another (thus forming a double bond between their immediately adjacent carbon atoms);

R₈, R₉ are the same or different and are either hydrogen or together form an oxo radical;

R₁₀ is either H or form bond with one another (thus forming a double bond between its immediately adjacent carbon atoms);

or a pharmaceutically acceptable salt or prodrug thereof.

A further aspect of the present invention is a composition comprising a compound of Formula (I) (an active compound) in a pharmaceutically acceptable carrier (such as an aqueous carrier).

A further aspect of the present invention is a composition comprising a compound of Formula (I) (an active compound) in a pharmaceutically acceptable carrier (such as an aqueous carrier) and one or more HIV entry inhibitors.

A further aspect of the present invention is directed to methods for treating a viral infection, particularly a retroviral infection (e.g., HIV-1 infection) in cells or tissue of an animal, in an animal subject or human, comprising administering an effective retroviral inhibiting amount of a compound of Formula (I). The examples of HIV infection includes, but not limit to, DSB-resistant HIV-1 infection and RPR103611-resistant HIV-1 infection, etc.

Another aspect of the present invention is that methods of treating a viral infection further comprise concurrently administering another HIV entry inhibitor.

### BRIEF DESCRIPTION OF THE DRAWINGS

FIG. 1: Betulinic acid derivatives inhibited HIV-1 envelope mediated membrane fusion. FIG. 1 illustrates the anti-fusion activity of [[N-[3β-O-(3′,3′-Dimethylsuccinyl)-lup-20 (29)-en-28-oyl]-7-aminoheptyl]-carbamoyl]methane (18) and [[N-[3β-Hydroxy-lup-20 (29)-en-28-oyl]-7-aminoheptyl]-carbamoyl]methane (18b). Compound 18 is represented by A12-2 and compound 18 b is represented by A 43-D. Each data point represents the mean+/−SD of two duplicated experiments.

FIG. 2: Betulinic acid derivatives interfere with HIV-1 p25 processing. FIG. 2 illustrates anti-maturation activity of [[N-[3β-O-(3′,3′-Dimethylsuccinyl)-lup-20(29)-en-28-oyl]-7-aminoheptyl]-carbamoyl]methane (18). Compound 18 is represented by A12-2.

### DETAILED DESCRIPTION OF THE PREFERRED EMBODIMENTS

“Acyl” as used herein means a —C(O)R radical, where R is a suitable substituent.

“Alkyl” as used herein, refers to a straight or branched chain hydrocarbon, preferably containing from 1 to 10 carbon atoms. In some embodiments, alkyl group has 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, isopropyl, n-butyl, iso-butyl, tert-butyl, and the like.

“Aryl” as used herein, refers to a monocyclic carbocyclic ring system or a bicyclic carbocyclic fused ring system having one or more aromatic rings. Representative examples of aryl include azulenyl, indanyl, indenyl, naphthyl, phenyl, tetrahydronaphthyl, and the like. Unless otherwise indicated, the aryl groups of this invention can be unsubstituted or substituted with one or more suitable substituents.

“Acyl carboxylic acid” as used herein, refers to an organic group of —C(O)—R—COOH, wherein R is any organic group such as alkyl, aryl, alkylaryl, optionally substituted 1, 2, 3 times or more with independently selected, hetero atoms such as O, N, S, halogen atoms such as F, Cl, Br, I, hydroxy, alkyl, haloalkyl, alkoxy, haloalkoxy, cycloalkyl, or heterocycle. The examples of suitable acyl carboxylic acid groups include but are not limited to those shown as R₁ group in U.S. Pat. No. 6,172,110 to Lee et al., the disclosure of which is incorporated by reference herein in its entirety.

“Benzotriazole” as used herein, refers to a heteroaryl group of formula (A).

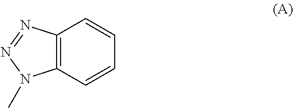

The benzotriazole groups of this invention can be unsubstituted or substituted with 1, 2, 3, or 4 suitable substituents independently selected from alkyl, alkenyl, alkenyloxy, alkoxy, alkoxycarbonyl, alkylcarbonyl, alkylcarbonyloxy, alkylsulfinyl, alkylsulfonyl, alkylthio, alkynyl, aryl, aryloxy, azido, arylalkoxy, arylalkyl, aryloxy, carboxy, cyano, formyl, halogen, haloalkyl, haloalkoxy, hydroxy, hydroxyalkyl, mercapto, nitro, sulfamyl, sulfo, sulfonate, —NR′R″ (wherein, R′ and R″ are independently selected from hydrogen, alkyl, alkylcarbonyl, aryl, arylalkyl and formyl), and —C(O)NR′R″ (wherein R′ and R″ are independently selected from hydrogen, alkyl, alkylcarbonyl, aryl, arylalkyl, and formyl).

“Carboxy” as used herein refers to the radical —C(O)OH.

“Cycloalkyl” as used herein, refers to a saturated monocyclic or bicyclic hydrocarbon group containing from 3 to 12 carbons. Representative examples of cycloalkyl include cyclopropyl, cyclobutyl, cyclopentyl, cyclohexyl, cycloheptyl, cyclooctyl, naphthalene, bicycle-octane and bicyclo-nonane. Unless otherwise indicated, the cycloalkyl groups of this invention can be unsubstituted or substituted with one or more suitable substituents.

“Heteroaryl” as used herein refers to aromatic radicals containing from five to twenty skeletal ring atoms and where one or more of the ring atoms is a heteroatom such as, for example, oxygen, nitrogen, sulfur, selenium and phosphorus. The term heteroaryl includes optionally substituted mono-heteroaryl radicals and fused heteroaryl radicals having at least one heteroatom (e.g., quinoline, benzothiazole). A fused heteroaryl radical may contain from two to four fused rings and where the ring of attachment is a heteroaromatic ring, the other individual rings within the fused ring system may be aromatic, heteroaromatic, alicyclic or heterocyclic. The term heteroaryl also includes mono-heteroaryls or fused heteroaryls having from five to twelve skeletal ring atoms, as well as those having from five to ten skeletal ring atoms. Examples of heteroaryls include, without limitation, furanyl, benzofuranyl, chromenyl, pyridyl, pyrrolyl, indolyl, quinolinyl, pyridyl-N-oxide, pyrimidyl, pyrazinyl, imidazolyl, pyrazolyl, oxazolyl, isoxazolyl, benzothiozole, benzimidazole, benzoxazoles, benzothiadiazole, benzoxadiazole, benzotriazole, quinolines, isoquinolines, indolyl, purinyl, indolizinyl, thienyl and the like and their oxides. Unless otherwise indicated, the heteroaryl groups of this invention can be unsubstituted or substituted with one or more suitable substituents.

“Heterocycle” as used herein, refers to a monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5-membered ring has from 0-2 double bonds and the 6-membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like. Unless otherwise indicated, the heteroaryl groups of this invention can be unsubstituted or substituted with one or more suitable substituents.

The term “substituent” or “suitable substituent” herein refers to any suitable substituent that may be recognized or selected, such as through routine testing, by those skilled in the art. Illustrative examples of suitable substituents include alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, aryloxy, azido, arylalkoxy, arylalkyl, aryloxy, carboxy, cyano, formyl, halogen, haloalkyl, haloalkoxy, hydroxy, hydroxyalkyl, mercapto, nitro, sulfamyl, sulfo, sulfonate, —NR′R″ (wherein, R′ and R″ are independently selected from hydrogen, alkyl, alkylcarbonyl, aryl, arylalkyl and formyl), and —C(O)NR′R″ (wherein R′ and R″ are independently selected from hydrogen, alkyl, alkylcarbonyl, aryl, arylalkyl, and formyl).

“Treat” as used herein refers to any type of treatment that imparts a benefit to a patient afflicted with a disease, including improvement in the condition of the patient (e.g., in one or more symptoms), delay in the progression of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

A “prodrug” as used herein means a compound that is converted under physiological conditions or by solvolysis or metabolically to a specified compound that is pharmaceutically active.

“Concurrently administer” as used herein means that the two compounds or agents are administered closely enough in time to produce a combined effect (that is, concurrently may be simultaneously, or it may be two or more events occurring within a short time period before or after each other, e.g., sequentially). Simultaneous administration may be carried out by mixing the compounds prior to administration, or by administering the compounds at the same point in time but at different anatomic sites and/or by using different routes of administration.

All publications, U.S. patent applications, U.S. patents and other references cited herein are incorporated by reference in their entireties.

### 1. Active Compounds.

The methods of the present invention include the administration of active compounds as described herein (e.g., compounds of Formula (I)), while pharmaceutical compositions of the present invention comprise active compounds in a pharmaceutically acceptable carrier or diluent.

Active compounds of the present invention include compounds of Formula (I) or Formula (I′) as follows:

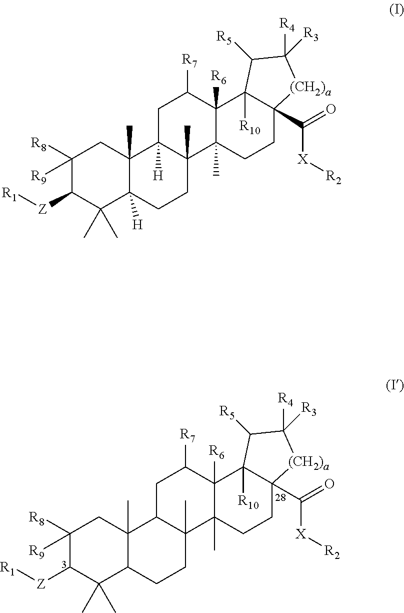

wherein:
a is 1 or 2;
Z is O, S, NH, or N-alkyl;
R₁ is a hydrogen,
or acyl carboxylic acid,
or R₁ is a substituent of the formula

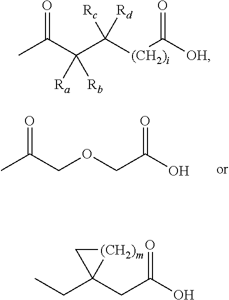

wherein Rₐ, R_{b}, R_{c} and R_{d} are the same or different and are each independently selected from the group consisting of hydrogen or lower alkyl, i is an integer from 0 to 3, and m is an integer from 1 to 4;
X is O, S, NH, or N-alkyl;
R₂ is a substituent of the formula:

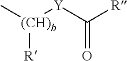

wherein:
   R′ and R″ are either hydrogen or alkyl radicals;
   Y is O, S, NH, N-alkyl, or heterocycle (e.g. piperazine);
   b is an integer from 0 to 16;

or R₇ is benzotriazole;

R₃ and R₄ are either H or lower alkyl;

R₅ is H, lower alkyl or —CRᵢRᵢᵢRᵢᵢᵢ,

Rᵢ is a methyl radical or forms with Rᵢᵢᵢ a methylene radical or an oxo radical,

Rᵢᵢ is a hydroxyl, methyl or hydroxymethyl radical or a radical —CH₂ OR′ᵢᵢ, —CH₂SR′ᵢᵢ or —CH₂NHR′ᵢᵢ for which R′ᵢᵢ is alkyl, hydroxyalkyl, dihydroxyalkyl, acetamidoalkyl or acetyl, or Rᵢᵢ is an amino radical substituted with a hydroxyalkyl or carboxyhydroxyalkyl radical, or a dialkylamino radical, the alkyl parts of which can form, with the nitrogen atom to which they are joined, a 5- or 6-membered heterocycle optionally containing another hetero atom chosen from oxygen, sulphur or nitrogen and, optionally, N-alkyl;

Rᵢᵢᵢ is a hydrogen atom or forms, with Rᵢ or Rᵢᵢ, a methylene radical or an oxo radical,

or R₅ form a bond with its immediately adjacent carbon atoms;

R₆, R₇ are the same or different and are either H or form bond with one another (thus forming a double bond between their immediately adjacent carbon atoms);

R₈, R₉ are the same or different and are either hydrogen or together form an oxo radical;

R₁₀ is either H or form bond with one another (thus forming a double bond between its immediately adjacent carbon atoms);

or a pharmaceutically acceptable salt or prodrug thereof.

Illustrative embodiments of compounds of Formula (I) or Formula (I′) include compounds of Formula (Ia), Formula (Ib), and Formula (Ic) below:

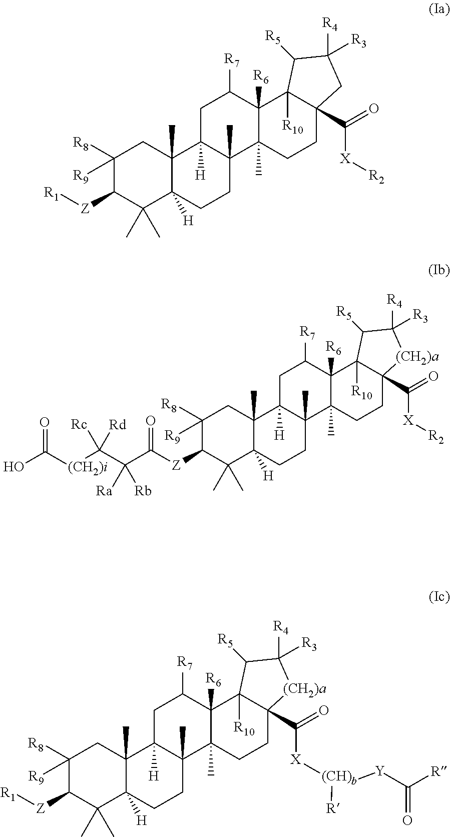

In which the substituents are defined in connection with Formula (I) above.

In some preferred embodiments of compounds of Formula (I), (I′), (Ia), (Ib) or (Ic) above, R₂ is a substituent of the formula:

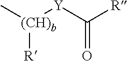

wherein:
R′ and R″ are either hydrogen or alkyl radicals,
Y is O, S, NH, N-alkyl or heterocycle,
b is an integer from 0 to 16.

In some preferred embodiments of compounds of Formula (I), (I′), (Ia), (Ib) or (Ic) above, R₁ is a hydrogen,

or a substituent of the formula

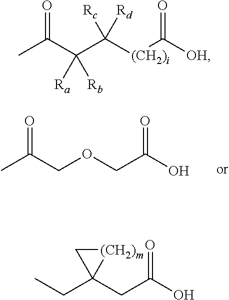

wherein Rₐ, R_{b}, R_{c} and R_{d} are the same or different and are each independently selected from the group consisting of hydrogen or lower alkyl, i is an integer from 0 to 3, and m is an integer from 1 to 4.

In some preferred embodiments of compounds of Formula (I), (I′), (Ia), (Ib) or (Ic) above, R₆ and R₁₀ are each H.

In some preferred embodiments of compounds of Formula (I), (I′), (Ia), (Ib) or (Ic) above, R₁ is

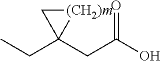

and m is an integer from 1 to 4.
In some preferred embodiments of compounds of Formula (I), (I′), (Ia), (Ib) or (Ic) above, R₅ is —CRᵢRᵢᵢRᵢᵢᵢ.

In some preferred embodiments of compounds of Formula (I), (I′), (Ia), (Ib) or (Ic) above, R₈ and R₉ are each H.

In some preferred embodiments of compounds of Formula (I), (I′), (Ia), (Ib) or (Ic) above, Rᵢ and Rᵢᵢᵢ together form a methylene radical.

In some preferred embodiments of compounds of Formula (I), (I′), (Ia), (Ib) or (Ic) above, Rᵢᵢ is methyl.

In some preferred embodiments of compounds of Formula (I), (I′), (Ia), (Ib) or (Ic) above, R₃ and R₄ are each H.

In some preferred embodiments of compounds of Formula (I), (I′), (Ia), (Ib) or (Ic) above, R₆ and R₇ are each H.

In some preferred embodiments of compounds of Formula (I), (I′), (Ia), (Ib) or (Ic) above, the compound has the following formula:

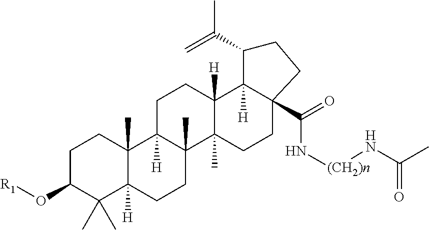

wherein R₁ is either H or

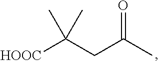

and n is an integer from 1-10, or a pharmaceutically acceptable salt or prodrug thereof.

Illustrative compounds of some embodiments of the present invention are set forth below:

[[N-[3β-O-(3′,3′-Dimethylsuccinyl)-lup-20(29)-en-28-oyl]-6-aminopenyl]-carbamoyl]methane (16)

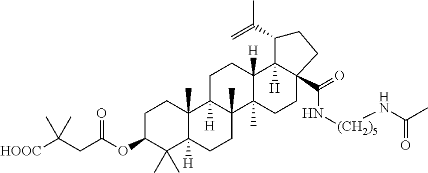

[[N-[3β-O-(3′,3′-Dimethylsuccinye-lup-20(29)-en-28-oyl]-6-aminohexyl]-carbamoyl]methane (17)

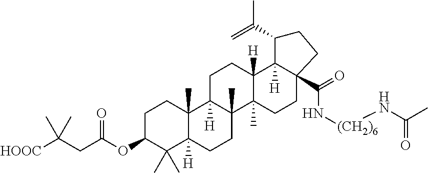

[[N-[3β-O-(3′,3′-Dimethylsuccinyl)-lup-20(29)-en-28-oyl]-7-aminoheptyl]-carbamoyl]methane (18)

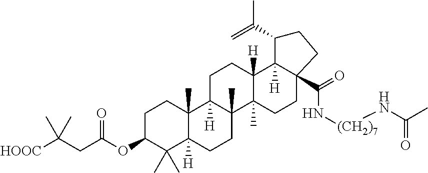

[[N-[3β-O-(3′,3′-Dimethylsuccinyl)-lup-20(29)-en-28-oyl]-8-aminooctyl]-carbamoyl]methane (19)

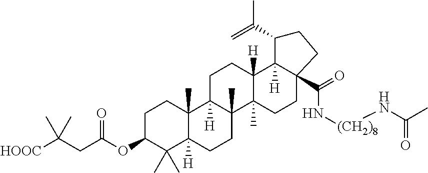

[[N-[3β-O-(3′,3′-Dimethylsuccinyl)-lup-20(29)-en-28-oyl]-9-aminonoyl]-carbamoyl]methane (20)

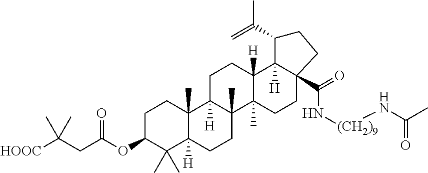

O-[3β-O-(2′,2′-Dimethylsuccinyl)-lup-20(29)-en-28-oyl]-1-hydroxybenzotriazole (11)

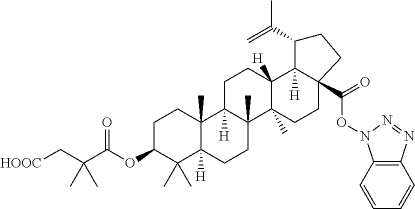

[[N-[3β-Hydroxy-lup-20(29)-en-28-oyl]-7-aminoheptyl]-carbamoyl]methane. (18b)

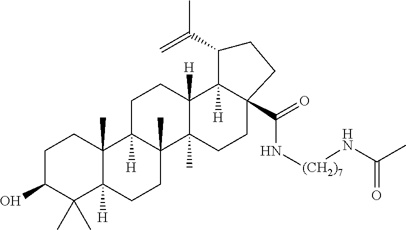

Compounds of the present invention (i.e., active compounds) can be made in accordance with Scheme I, and/or Scheme II below, or variations thereof that will be obvious to those skilled in the art in light of the disclosure herein.

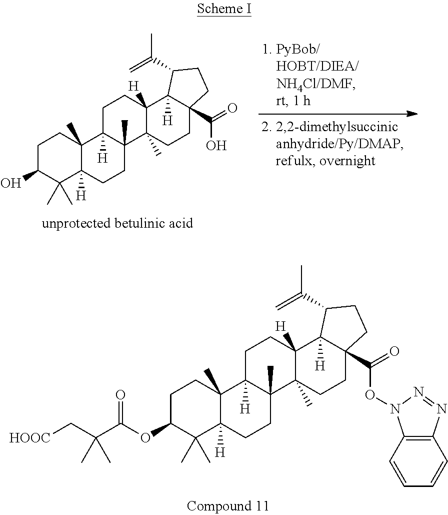

Compound II is O-[3β-O-(2′,2′-Dimethylsuccinyl)-lup-20(29)-en-28-oyl]-1-hydroxybenzotriazole.

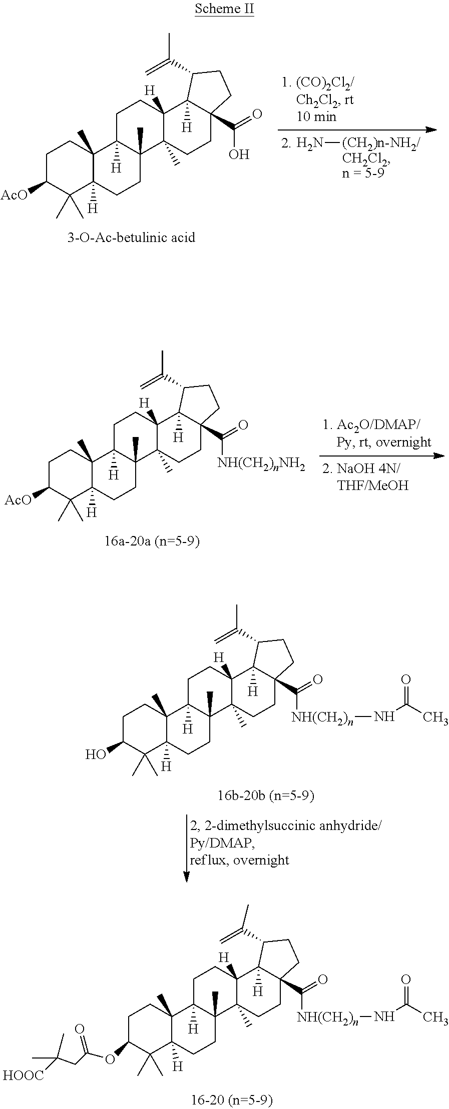

Compound 16 is [[N-[3β-O-(3′,3′-dimethylsuccinyl)-lup-20(29)-en-28-oyl]-5-aminopentyl]-carbamoyl]methane.

Compound 17 is [[N-[3β-O-(3′,3′-Dimethylsuccinyl)-lup-20(29)-en-28-oyl]-6-aminohexyl]-carbamoyl]methane.

Compound 18 is [[N-[3β-(3′,3′-Dimethylsuccinyl)-lup-20(29)-en-28-oyl]-7-aminoheptyl]-carbamoyl]methane.

Compound 18b is [[N-[3β-Hydroxy-lup-20 (29)-en-28-oyl]-7-aminoheptyl]-carbamoyl]methane.

Compound 19 is [[N-[3β-O-(3′,3′-Dimethylsuccinyl)-lup-20(29)-en-28-oyl]-8-aminooctyl]-carbamoyl]methane.

Compound 20 is [[N-[3β-O-(3′,3′-Dimethylsuccinyl)-lup-20(29)-en-28-oyl]-9-aminonoyl]-carbamoyl]methane

The active compounds disclosed herein or described above can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active compounds may be provided as pharmaceutically acceptable prodrugs, which are those prodrugs of the active compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

### 2. Pharmaceutical Formulations

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formula (I) or Formula (I′), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 1 or 10 mg to about 100 milligrams, 1 gram or 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the farm of an optionally buffered aqueous solution of the active compound. Suitable formulations comprise citrate or bis\tris buffer (pH 6) or ethanol/water and contain from 0.1 to 0.2M active ingredient.

In addition to compounds of Formula (I) or Formula (I′) or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### 3. Methods of Treatment

The present invention is primarily concerned with the treatment of human subjects, but the invention may also be carried out on animal subjects, particularly mammalian subjects such as mice, rats, dogs, cats, non-human primates, livestock and horses for veterinary purposes, and for drug screening and drug development purposes.

Examples of retroviral infections that may be treated by the methods of the present invention include but are not limited to feline leukemia virus (FeLV), human immunodeficiency virus (HIV; including both HIV-1 and HIV-2) simian immunodeficiency virus (SIV) and other lentiviral infections such as equine infectious anemia virus (EAIV) and feline immunodeficiency virus (FIV). A particularly preferred embodiment is use of the methods, compounds and compositions of the present invention for the treatment of HIV-1 infection in human subjects.

In one embodiment of the present invention, the methods may be used to treat HIV-1 infections that are resistant to treatment with DSB or a pharmaceutically acceptable salt thereof and/or resistant to treatment with RPR103611 or a pharmaceutically acceptable salt thereof (these two compounds illustrated below).

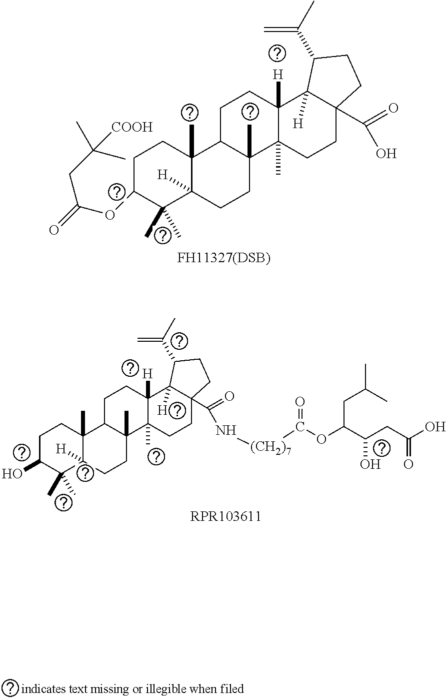

By “resistant” is meant that the efficacy of the compound to which resistance has developed is significantly reduced as compared to infections which are not resistant to treatment with the same compound or compounds.

Without wishing to be bound to any particular theory of the instant invention, it is believed (and preferred) that compounds of the present invention interfere with two different and distinct steps of the HIV-1 life cycle: first, compounds of the present invention inhibit the early step of HIV-1 entry into cells; second, compounds of the present invention inhibit HIV-1 maturation (e.g., by inhibiting the last step of HIV-1 gag processing, the cleavage between P24 and p2 required for HIV-1 maturation). The pharmacophore believed responsible for the anti-entry activity is believed to reside at the 3 position and the side chain substituted thereon, and the pharmacophore believed responsible for the anti-maturation activity is believed to reside at the 28 position and side-chain substituted thereon.

### 4. Dosage and Routes of Administration

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. Typical dosages comprise at about 0.1 to about 100 mg/kg body weight. One preferred dosages comprise about 1 to about 100 mg/kg body weight of the active ingredient. One still more preferred dosages comprise about 10 to about 100 mg/kg body weight.

### 5. Combination Methods and Compositions

Methods of treatment as described herein can include concurrently administering one or more additional anti-viral agent (including HIV entry inhibitor discussed below), and compositions as described herein can optionally include one or more such additional antiviral agents. Examples of such additional antiviral agents include, but are not limited to, AZT (Glaxo Wellcome), 3TC (Glaxo Wellcome), ddI (Bristol-Myers Squibb), ddC (Hoffmann-La Roche), D4T (Bristol-Myers Squibb), abacavir (Glaxo Wellcome), nevirapine (Boehringher Ingelheim), delavirdine (Phamiacia and Upjohn), efavirenz (DuPont Pharmaceuticals), saquinavir (Hoffmann-La Roche), ritonavir (Abbott Laboratories), indinavir (Merck and Company), nelfinavir (Agouron Pharmaceuticals), amprenavir (Glaxo Wellcome), adefovir (Gilead Sciences), hydroxyurea (Bristol-Meyers Squibb), AL-721 (lipid mixture) manufactured by Ethigen Corporation and Matrix Research Laboratories; Amphotericin B methyl ester; Ampligen (mismatched RNA) developed by DuPont/HEM Research; anti-AIDS antibody (Nisshon Food); 1 AS-101 (heavy metal based immunostimulant); Betaseron (.beta.-interferon) manufactured by Triton Biosciences (Shell Oil); butylated hydroxytoluene; Carrosyn (polymannoacetate); Castanospermine; Contracan (stearic acid derivative); Creme Pharmatex (containing benzalkonium chloride) manufactured by Pharmalec; CS-87 (5-unsubstituted derivative of Zidovudine), Cytovene (ganciclovir) manufactured by Syntex Corporation; dextran sulfate; D-penicillamine (3-mercapto-D-valine) manufactured by Carter-Wallace and Degussa Pharmaceutical; Foscarnet (trisodium phosphonoformate) manufactured by Astra AB; fusidic acid manufactured by Leo Lovens; glycyrrhizin (a constituent of licorice root); HPA-23 (ammonium-21-tungsto-9-antimonate) manufactured by Rhone-Poulenc Sante; human immune virus antiviral developed by Porton Products International; Ornidyl (eflornithine) manufactured by Merrell-Dow; nonoxinol; pentamidine isethionate (PENTAM-300) manufactured by Lypho Med; Peptide T (octapeptide sequence) manufactured by Peninsula Laboratories; Phenyloin (Warner-Lambert); Ribavirin; Rifabutin (ansamycin) manufactured by Adria Laboratories; CD4-IgG2 (Progenics Pharmaceuticals) or other CD4-containing or CD4-based molecules; T-20 (Trimeris); Trimetrexate manufactured by Warner-Lambert Company; SK-818 (germanium-derived antiviral) manufactured by Sanwa Kagaku; suramin and analogues thereof manufactured by Miles Pharmaceuticals; UA001 manufactured by Ueno Fine Chemicals Industry; and alpha-interferon, manufactured by Glaxo Wellcome.

HIV entry inhibitors are a class of anti HIV drugs that work by preventing HIV from entering susceptible cells in the body. In generally, it is preferred that the HIV entry inhibitor (1) block virus entry into susceptible cells by preventing HIV-1 binding to the cellular receptor CD4, the coreceptors CXCR4/CCR5 and to receptors on dendritic/migratory cells (capturing and transmitting virus to cells which are directly involved in virus replication), respectively. (See The entry of entry inhibitors: a fusion of science and medicine, Moore, J. P, etc, Proc. Natl. Acad. Sci., USA, 100, 10598-10602, (2003); HIV-1 entry inhibitors: new targets, novel therapies, Pierson, T. C., etc., Immunol. Lett., 85, 113-118, (2003); HIV Transmission: Closing all the Doors, Davis, C. W., etc, J. Exp. Med, 199, 1037-1040, (2004); Blockade of attachment and fusion receptors inhibits HIV-1 infection of human cervical tissue, Hu, Q., Frank, etc, J. Exp. Med., 199, 1065-1075, (2004)), and/or (2) are virucidal.)

Examples of HIV inhibitors include but not limited to: CCR5 inhibitors TAK-779, Fusion inhibitors T20, CXCR4 inhibitor AMD 3100, and other inhibitors BMS 378806, etc. The structures of representative HIV entry inhibitors are illustrated below.

The chemical structure of TAK-779, N,N-dimethyl-N-(4[[[2-(4-methylphenyl)-6,7-dihydro-5H-benzocyclohepten-8-yl]carbon-yl]benzyl]-tetrahydro-2H-pyran, is shown below. (See Structure Modeling of the Chemkine Receptor CCR5: Implications for Ligand Binding and Selectivity, M. Germana Paterlini, Biophysical Journal, 83, 3012-3031 (2002).)

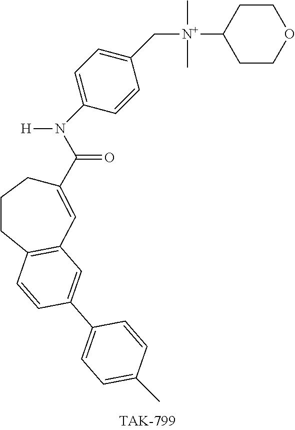

T20 (FUZEON™ or enfuvirtide) is a linear 36-amino acid synthetic peptide with an acetylated N-terminus and a carboxamide C-terminus. It is composed of naturally occurring L-amino acid residues. The empirical formula of enfuvirtide is C₂₀₄H₃₀₁N₅₁O₆₄. It has the following primary amino acid sequence: CH₃CO-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-Leu-Asp-Lys-Trp-Ala-Ser-Leu-Trp-Asn-Trp-Phe-NH2 and the following structural formula:

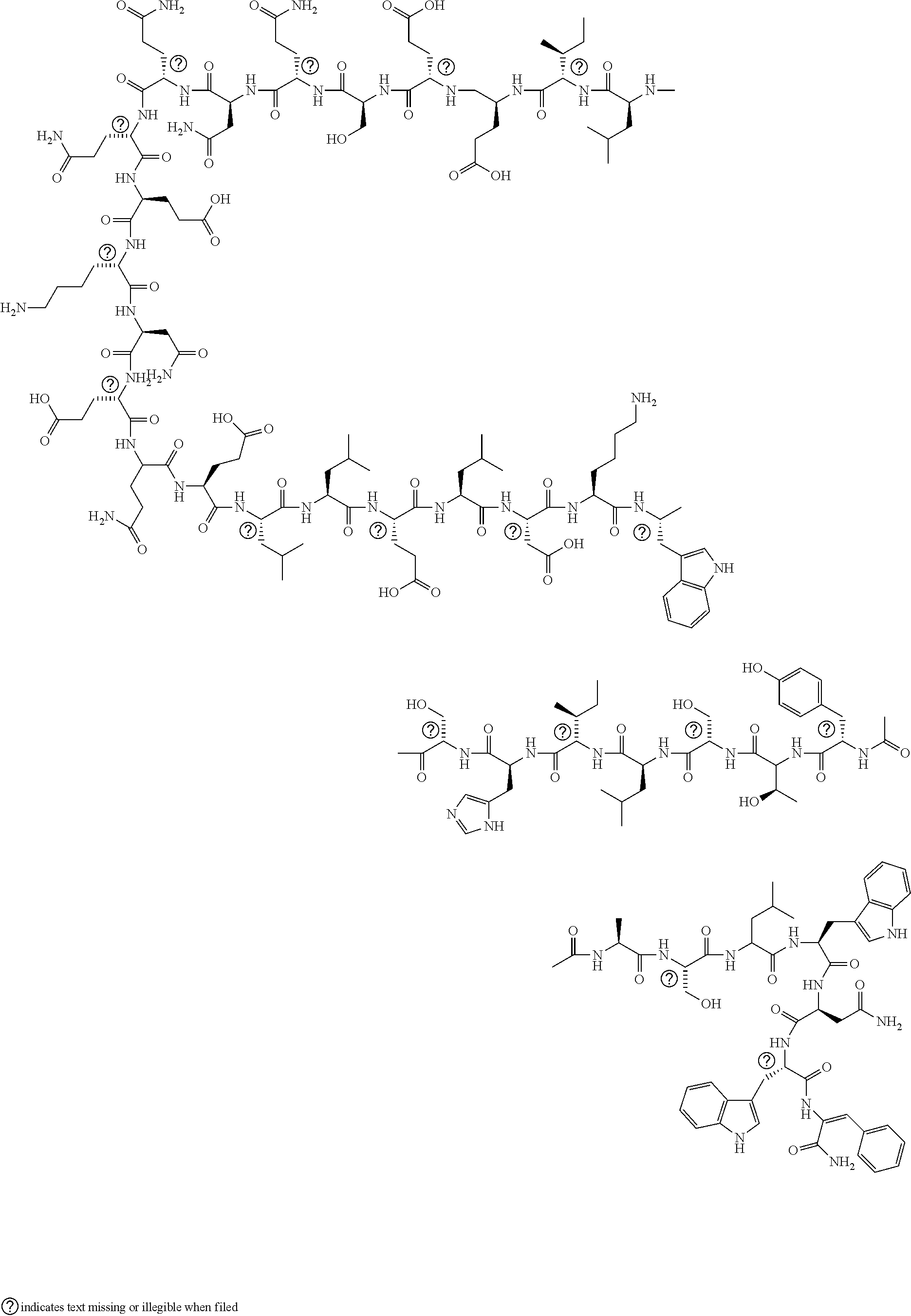

The chemical structure of BMS-378806 (2R)-4-benzoyl-1-[2-(4-methoxy-1H-pyrrolo[2,3-b]pyridin-3-yl)-1,2-dioxoethyl]-2-methyl-piperazine, is shown below (See A small molecule HIV-1 inhibitor that targets the HIV-1 envelope and inhibits CD4

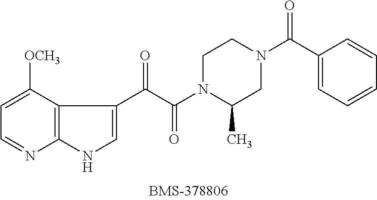

receptor binding, Pin-Fang Lin, et al., PNAS, 100, 19, 1103-11018 (2003, September).)

The chemical structure of AMD 3100, 1,1′-[1,4-phenylenebis(methylene)]-bis-1,4,8,11-tetraazacyclotetradecane is shown below. (See AMD 3100, a Potent and Specific Antagonist of the Stromal Cell-Derived Factor-1 Chemokine Receptor CXCR4, Inhibits Autoimmune Joint Inflammation in IFN-γ Receptor-Deficient Mice, Patrick Matthys, etc, The Journal of Immunology, 167, 4686-4692. (2001).)

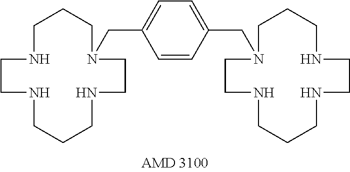

The compounds of the present invention may be concurrently administered in combination with one or more HIV entry inhibitors for the treatment of a mammal, such as a human, that is suffering from an infection with the HIV virus, AIDS, AIDS-related complex, or any disease or condition which is related to infection with the HIV virus.

Pharmaceutical compositions of the present invention can also further comprise immunomodulators, and methods of treatment of the present invention can include the co-administration of an immunomodulator. Suitable immunomodulators for optional use with the active compounds of the present invention in accordance with the present invention can include, but are not limited to: ABPP (Bropririmine); Ampligen (mismatched RNA) DuPont/HEM Research; anti-human interferon-.alpha.-antibody (Advance Biotherapy and Concepts); anti-AIDS antibody (Nisshon Food); AS-101 (heavy metal based immunostimulant; ascorbic acid and derivatives thereof; interferon-.beta.; Carrosyn (polymannoacetate); Ciamexon (Boehringer-Mannheim); cyclosporin; cimetidine; CL-246,738 (American Cyanamid); colony stimulating factors, including GM-CSF (Sandoz, Genetics Institute); dinitrochlorobenzene; HE2000 (Hollis-Eden Pharmaceuticals); interferon-.alpha.; inteferon-gamma; glucan; hyperimmune gamma-globulin (Bayer); IMREG-1 (leukocyte dialyzate) and IMREG-2 (IMREG Corp.); immuthiol (sodium diethylthiocarbamate) (Institut Merieux); interleukin-1 (Cetus Corporation; Hoffmann-LaRoche; Immunex); interleukin-2 (IL-2) (Chiron Corporation); isoprinosine (inosine pranobex); Krestin (Sankyo); LC-9018 (Yakult); lentinan (Ajinomoto/Yamanouchi); LF-1695 (Fournier); methionine-enkephalin (TNI Pharmaceuticals; Sigma Chemicals); Minophagen C; muramyl tripeptide, MTP-PE (Ciba-Geigy); naltrexone (“Trexan” DuPont); Neutropin, RNA immunomodulator (Nippon Shingaku); Remune (Immune Response Corporation); Reticulose (Advanced Viral Research Corporation); shosaikoto and ginseng; thymic humoral factor; TP-05 (Thymopentin, Ortho Pharmaceuticals); Thymosin factor 5 and Thymosin 1; Thymostimulin; TNF (Tumor necrosis factor) manufactured by Genentech; and vitamin B preparations.

Further embodiments of the present invention will now be described with reference to the following examples. It should be appreciated that these examples are for the purposes of illustrating embodiments of the present invention, and do not limit the scope of the invention.

### EXAMPLES

### Chemical Examples

General experiment procedures: All melting points were determined with a Fisher-Johns melting point apparatus without correction. Positive and negative HR-FABMS were recorded on a Joel SX-102 spectrometer. ¹H and other NMR spectra were measured on a Varian Mercury 300 or Varian Inova 500 or Bruker DRX-400 spectrometer. Other than as noted, all samples were dissolved in CDCl₃ with TMS as internal standard. Si gel chromatography was carried out on a Biotage Horizon Flash chromatograph system with prepacked Si gel column. HPLC was performed on a Varian ProStar solvent delivery and PDA detector with Agilent Zorbax ODS or C-8 columns (4.6 mm×25 cm and 9.4 mm×25 cm for analytical and semi preparative scale, respectively).

As used herein, “DMF” refers to dimethylformamide, “DMAP” refers to 4-Dimethylaminopyridine and “Py” refers to pyridine.

General methods and experimental for preparing compounds of the present invention are set forth below. In certain cases, a particular compound is described by way of example. However, it will be appreciated that in each case a series of compounds of the present invention were prepared in accordance with the schemes described above and examples described below.

### Example 1

### [[N-[3β-O-(3′,3′-Dimethylsuccinyl)-lup-20(29)-en-28-oyl]-6-aminopenyl]-carbamoyl]methane (16)

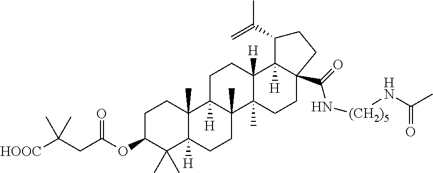

A reaction mixture of 3-O-acyl betulinic acid (300 mg, 0.6 mmol) and oxalyl chloride (6 mmol, 2 M in CH₂Cl₂) was stirred for 10 minutes, then the reaction mixture was dried under vacuum. The residue was dissolved in dichloromethane, and then the residue was slowly added to a solution of a 1,5-pentanediamine (400 mg, 2.5˜3.9 mmol) in dichloromethane (4 mL). After stirring overnight, the reaction mixture was concentrated, washed with water, then, dissolved in EtOH, and filtered to remove the insoluble solid. Then, the EtOH solution was concentrated under vacuum, and the residue was purified by silicon gel chromatography to yield the corresponding amine intermediates 16a.

After dissolving the amine intermediate 16a in anhydrous pyridine (4 mL), acetic anhydride (2 eq.) and DMAP (1 eq.) were added. After stirring overnight at room temperature, the reaction mixture was concentrated under vacuum. The residue was purified by a silicon gel chromatography to yield the corresponding C-28 mono-substituted betulinic acid derivatives. To the solution of mono-substituted betulinic acid derivative in THF/MeOH (50%, 4 mL), aqueous NaOH (1 mL, 4 M) was added. After stirring for 4 to 10 hours at room temperature, the mixture was neutralized with 1 N HCl. The resulting precipitation was collected, washed with water, and dried under vacuum to yield the corresponding C-28 mono-modified intermediates 16. Then, a mixture of C-28 mono-modified intermediates 16, 2,2-dimethylsuccinic anhydride (5 ˜10 eq.), and DMAP (1 eq.) in pyridine (anhydrous, 4 mL) was refluxed overnight. The reaction mixture was concentrated under vacuum, then, dissolved in MeOH. The MeOH solution was purified with reverse phase HPLC to yield [[N-[3β-O-(3′,3′-Dimethylsuccinyl)-lup-20(29)-en-28-oyl]-6-aminopenyl]-carbamoyl]methane (16). (Yield 27%) Mp 137° C. (dec). Positive FABMS m/z 711 (M+H)⁺; HR-FABMS calcd for C₄₃H₇₁N₂O₆ 711.5312, found 711.5313. ¹H NMR (500 MHz) δ 6.02 (1H, br s, NH), 5.67 (1H, t, J=6.0 Hz, NH), 4.72, 4.59 (each 1H, each s, ═CH₂), 4.48 (1H, dd, J=6.0 Hz, J=10.0 Hz, H-3), 3.16-3.32 (4H, m, 2×CH₂—NH), 3.12 (1H, dt, J=4.0 Hz, J=11.0 Hz, H-19), 2.56, 2.67 (each 1H, d, J=16.0 Hz, —CH—COO—CH), 2.43 (1H, dt, J=2.5 Hz, J=12.0 Hz, H-13), 2.01 (3H, s, COCH₃), 1.68 (3H, s, CH₃-30), 1.28, 1.30 (each 3H, s, —C(CH₃)₂—COOH), 0.80, 0.91, 0.96 (each 3H, s, 3×CH₃) 0.83 (6H, s, 2×CH₃).

### Example 2

### [[N-[3β-O-(3′,3′-Dimethylsuccinyl)-lup-20(29)-en-28-oyl]-6-aminohexyl]-carbamoyl]methane (17)

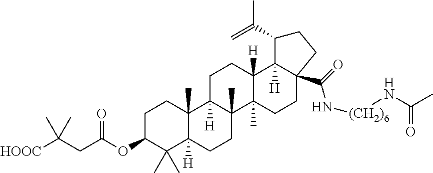

[[N-[3β-O-(3′,3′-Dimethylsuccinye-lup-20(29)-en-28-o yl]-6-aminohexyl]-carbamoyl]methane (17) were prepared by a similar manner as Example 1. Yield 17%. Mp 137° C. (dec). Positive FABMS m/z 725 (M+H)⁺; HR-FABMS calcd for C₄₄H₇₃N₂O₆ 725.5474, found 725.5469. ¹H NMR (300 MHz) δ 5.89 (1H, br s, NH), 5.70 (1H, t, J=6.0 Hz, NH), 4.70, 4.58 (each 1H, each s, ═CH₂), 4.47 (1H, dd, J=6.5 Hz, J=10.5 Hz, H-3), 3.17-3.29 (4H, m, 2×CH, —NH), 3.10 (1H, dt, J=3.5 Hz, J=14.0 Hz, H-19), 2.56, 2.66 (each 1H, d, J=16.0 Hz, —CH₂—COO—CH), 2.43 (1H, t, J=12.0 Hz, H-13), 1.99 (3H, s, COCH₃), 1.67 (3H, s, CH₃-30), 1.27, 1.29 (each 3H, s, —C(CH₃)₂—COOH), 0.79, 0.91, 0.94 (each 3H, s, 3×CH₃) 0.81 (6H, s, 2×CH₃).

### Example 3

### [[N-[3β-O-(3′,3′-Dimethylsuccinyl)-lup-20(29)-en-28-oyl]-7-aminoheptyl]-carbamoyl]methane (18)

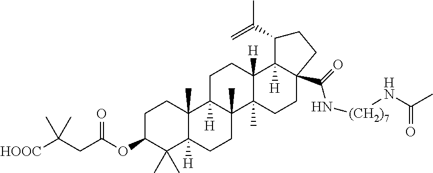

[[N-[3β-O-(3′,3′-Dimethylsuccinyl)-lup-20(29)-en-28-oyl]-7-aminoheptyl]-carbamoyl]methane (18) was prepared by a similar manner as Example 1. Yield 29%. Mp 117-120° C. Positive FABMS m/z 739 (M+H)⁺; HR-FABMS calcd for C₄₅H₇₅N₂O₆ 739.5625, found 739.5615. ¹H NMR (300 MHz) δ 5.62 (2H, t, J=5.0 Hz, 2×NH), 4.72, 4.59 (each 1H, each s, ═CH₂), 4.49 (1H, dd, J=6.5 Hz, J=10.0 Hz, 1′-3), 3.09-3.30 (5H, m, 2×CH₂—NH and H-19), 2.55, 2.67 (each 1H, d, J=16.0 Hz, —CH₂—COO—CH), 2.46 (1H, t, J=11.0 Hz, H-13), 1.99 (3H, s, COCH₃), 1.68 (3H, s, CH₃-30), 1.29, 1.30 (each 3H, s, —C(CH₃)₂—COOH), 0.80, 0.93, 0.96 (each 3H, s, 3×CH₃) 0.83 (6H, s, 2×CH₃).

### Example 4

### [[N-[3β-O-(3′,3′-Dimethylsuccinyl)-lup-20(29)-en-28-oyl]-8-aminooctyl]-carbamoyl]methane (19)

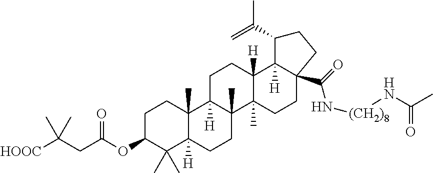

[[N-[3β-O-(3′,3′-Dimethylsuccinyl)-lup-20(29)-en-28-oyl]-8-aminooctyl]-carbamoyl]methane (19) was prepared by a similar manner as Example 1. Yield 59%. Mp113-115° C. Positive FABMS m/z 753 (M+H)⁺; HR-FABMS calcd for C₄₆H₇₇N₂O₆ 753.5781, found 753.5779. ¹H NMR (300 MHz) δ 5.83 (1H, br s, NH), 5.67 (1H, t, J=5.5 Hz, NH), 4.72, 4.58 (each 1H, each s, ═CH₂), 4.47 (1H, t, J=7.0 Hz, H-3), 3.06-3.29 (5H, m, 2×CH₂—NH and H-19), 2.55, 2.66 (each 1H, d, J=16.0 Hz, —CH—COO—CH), 2.43 (1H, t, J=10.0 Hz, H-13), 2.00 (3H, s, COCH₃), 1.67 (3H, s, CH₃-30), 1.27, 1.29 (each 3H, s, —C(CH₃)₂—COOH), 0.79, 0.91, 0.95 (each 3H, s, 3×CH₃) 0.82 (6H, s, 2×CH₃).

### Example 5

[[N-[3β-O-(3′,3′-Dimethylsuccinyl)-lup-20(29)-en-28-oyl]-9-aminonoyl]-carbamoyl]methane (20)

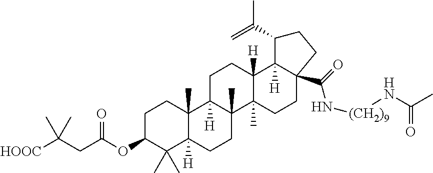

[[N-[3β-O-(3′,3′-Dimethylsuccinyl)-lup-20(29)-en-28-oyl]-9-aminonoyl]-carbamoyl]methane (20) was prepared by a similar manner as Example 1. Yield 40%. Mp 120-122° C. Positive FABMS m/z 698 (M+H)⁺; HR-FABMS calcd for C₄₇H₇₉N₂O₆ 767.5938, found 767.5944. ¹H NMR (300 MHz) δ 5.72 (1H, br s, NH), 5.64 (1H, t, J=5.5 Hz, NH), 4.72, 4.59 (each 1H, each s, ═CH₂), 4.48 (1H, t, J=7.0 Hz, H-3), 3.08-3.31 (5H, m, 2×CH₂—NH and H-19), 2.55, 2.67 (each 1H, d, J=16.0 Hz, —CH₂—COO—CH), 2.42 (1H, t, J=12.0 Hz, H-13), 2.00 (3H, s, COCH₃), 1.68 (3H, s, CH₃-30), 1.28, 1.29 (each 3H, s, —C(CH₃)₂—COOH), 0.80, 0.92, 0.95 (each 3H, s, 3×CH₃) 0.82 (6H, s, 2×CH₃).

### Example 6

### O-[3β-O-(2′,2′-Dimethylsuccinyl)-lup-20(29)-en-28-oyl]-1-hydroxybenzotriazole (11)

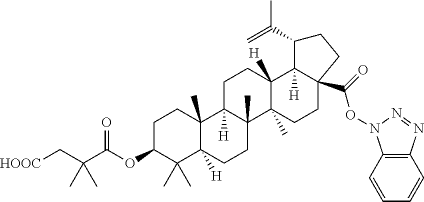

A mixture of betulinic acid (200 mg, 0.44 mmol), PyBop (343 mg, 0.66 mmol), HOBT (88 mg, 0.66 mmol), DIEA (0.26 mL, 1.76 mmol), and NH₄Cl (5 mg, 0.88 mmol) in DMF (4 mL) was stirred for 1 hour at room temperature. The mixture was then diluted with EtOAc (50 mL), washed with diluted HCl (1N) and brine, then dried over Na₂SO₄. The organic solution layer was then concentrated under vacuum and purifeid on a Silicon gel chromatography to yield the HOBT derivative of betulinic acid. Then, a mixture of above intermediate HOBT derivative of betulinic acid, 2,2-dimethylsuccinic anhydride (5 ˜10 eq.), and DMAP (1 eq.) in pyridine (anhydrous, 4 mL) was refluxed overnight. The reaction mixture was concentrated under vacuum, then, dissolved in MeOH. The MeOH solution was purified with reverse phase HPLC to yield O-[3β-O-(2′,2′-Dimethylsuccinyl)-lup-20(29)-en-28-oyl]-1-hydroxybenzotriazole (11) (Yield 64%). Mp 151-154° C. Negative FABMS m/z 700 (M−H)⁻; HR-FABMS calcd for C₄₂H₅₈N₃O₆ 700.4326, found 700.4326. ¹H NMR (300 MHz, Pyridine-d₅) δ 8.20 (1H, d, J=8.5 Hz, Ar—H), 7.67 (s, 1H, Ar—H), 7.64 (1H, dd, J=8.5 Hz, J=15.0 Hz, Ar—H), 7.42 (1H, dt, J=1.2 Hz, J=6.6 Hz, Ar—H), 4.88, 4.75 (each 1H, each s, ═CH₂), 4.71 (1H, d, J=4.7 Hz, H-3), 3.13 (1H, dd, J=4.7 Hz, J=11.0 Hz, H-19), 2.91 (2H, d, J=6.0 Hz, —CH₂—COOH), 2.65 (1H, d, J=2.8 Hz, CH), 2.38-2.47 (1H, m, CH), 2.31 (1H, t, J=12.0 Hz, H-13), 2.06-2.20 (1H, m, CH), 1.72 (3H, s, CH₃-30), 1.53 (6H, s, —C(CH₃)₂—CH₂—COOH), 0.71, 0.92, 0.95, 0.96, 1.02, (each 3H, s, 5×CH₃).

### Example 7

### [[N-[3β-Hydroxy-lup-20(29)-en-28-oyl]-7-aminoheptyl]-carbamoyl]methane. (18b)

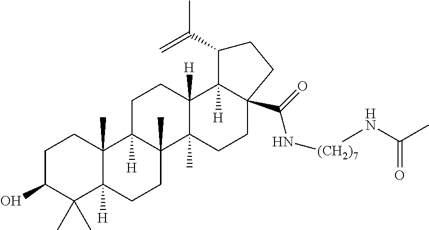

[[N-[3β-Hydroxy-lup-20(29)-en-28-oyl]-7-aminoheptyl]-carbamoyl]methane (18b) is the mono-functional derivative of 18 and was prepared by the similar manner as Example 1. 18b is isolated before C₃ coupling procedure is performed. HR-FABMS calcd for C₃₉H₆₇N₂O₃ 611.5252, found 611.5158. ¹H NMR (300 MHz) δ 5.50-5.63 (2H, m, 2×NH), 4.74, 4.59 (each 1H, each s, ═CH₂), 3.08-3.31 (6H, m, 2×CH₂—NH, H-3 and H-19), 2.47 (11-1, t, J=11.0 Hz, H-13), 1.98 (3H, s, COCH₃), 1.68 (3H, s, CH₃-30), 0.97 (6H, s, 2×CH₃), 0.94, 0.83, 0.76 (each 3H, s, 3×CH₃).

### Biological Assays

Anti-HIV assay: A previously known HIV-1 infectivity assay was used in the experiments (See Zhu, et al. Proc. Natl. Acad. Sci. U.S.A. 2001, 98, 15227). A 96-well microtiter plate was used to set up the HIV-1 NL4-3 replication assay. HIV-1 NL4-3 at a multiplicity of infection (MOI) of 0.01 was used to infect MT4 cells. Culture supernatants were collected on day 4 post infection for p24 assay using an ELISA kit from ZeptoMetrix Corporation (Buffalo, N.Y.).
Cell fusion assay: TZM cells that express luciferase upon fusion with envelope-expressing COS cells were used as fusion partners. The fusion assays were performed by transfecting monkey kidney cells (COS) with the expression vector pSRHS that contains the HIV-1 NL4-3 envelope genes. Electroporation was performed to express the HIV-1 envelope on COS cells. Briefly, COS cells (10⁶) in culture medium were incubated with 2 μg of the envelope expression vector on ice for 10 min. Electroporation was performed using a gene pulser (BioRad, Hercules, Calif.) with capacitance set at 950 μF and voltage at 150 V. The transfected COS cells were cultured for one day before mixing with TZM cells. TZM cells (10×10⁴) were incubated with COS cells (10⁴) in 96-well flat-bottomed plates (Costar) in 100 μl culture medium. Compounds to be tested at various concentrations in 10 μl of culture medium were incubated with the cell mixtures at 37° C. for 24 hours. A Promega luciferase assay kit was used to quantify luciferase activity in the fused cells using a Biotek luminometer. The drug concentration that reduced the control HIV-1 envelope-mediated fusion by 50% is defined as 50% effective concentration (EC₅₀).
HIV-1 maturation assay: MT4 cells (10⁵ cells/mL) were infected with HIV-1 NL4-3 at a multiplicity of infection (MOI) of 0.01 infectious units/cells for three days. The culture medium was removed and the infected cells were resuspended at 5×10⁵ cells/mL and treated with BA derivatives at various concentrations. The virions in the culture supernatant were collected one day after drug treatment. The viral particles were spun down and subjected to Western Blot analysis of p24 and p25 as previously described. (See Zhou, et al. J. Virol. 2004, 78, 922 and Zhou, et al., Retrovirology 2004, 1, 15.)

### Example 8

### Biological Activity

### Anti-Fusion Activity

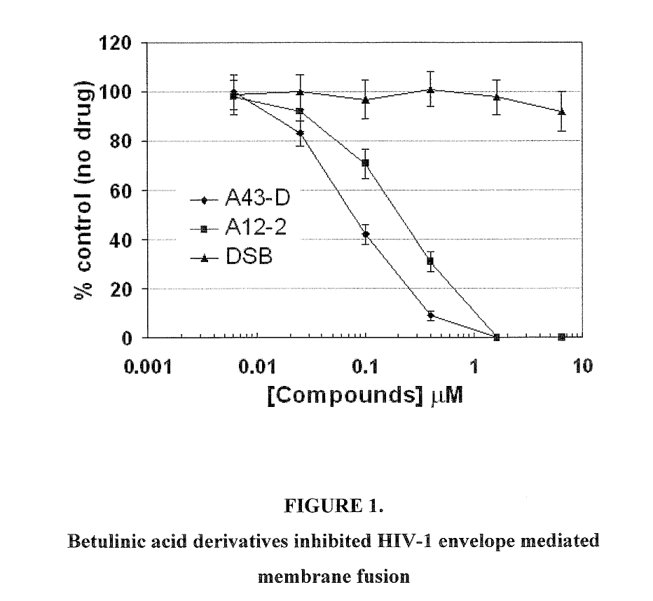

FIG. 1 graphically illustrates that betulinic acid derivatives (compounds 18 (also called as A12-2) and 18b (also called as A43-D)) inhibits HIV-1 envelope mediated membrane fusion. In FIG. 1, a luciferase-based fusion assay was used, and TZM cells that express luciferase upon fusion with envelope-expressing COS cells were used as fusion partners. The fusion assay performed in the absence of drugs is defined as 100% control fusion. In FIG. 1, each data point represents the mean+/−SD of two duplicated experiments.

### Example 9

### Biological Activity

### Anti-Maturation Activity

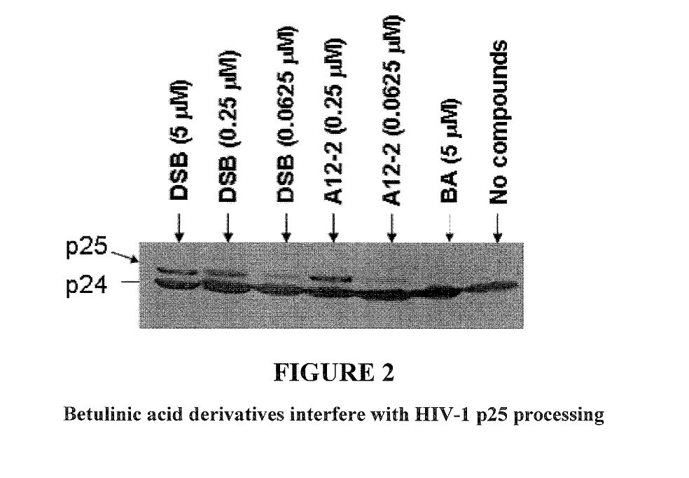

FIG. 2, betulinic acid derivatives interfere with HIV-1 p25 processing, graphically illustrated that the anti-maturation activity of compound 18, which is tested by detecting p25 using Western blot analysis. In FIG. 2, DSB at 5 μM was used as a marker for 100% inhibition, since DSB completely inhibited HIV NL4-3 replication at this concentration. Betulinic acid (BA) was used as a negative control, since it did not inhibit NL4-3 replication.

### Example 10

### Biological Activity

Table 1 illustrated the effect of compound 11, 16-20 and 18b against HIV-NL4-3 in MT4 cells.

TABLE 1

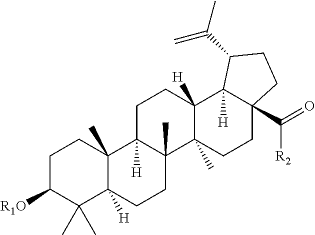

| Compound | R₁ | R₂ | EC₅₀(μM)^{a} | IC₅₀(μM)^{a} |
|---|---|---|---|---|
| 1 (IC9564) | H | 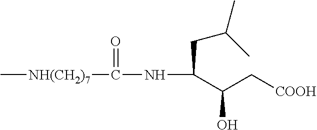 | 0.053 | >10 |
| 2 (DSB) | 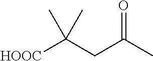 | OH | 0.075 | 7.5 |
| 11 | 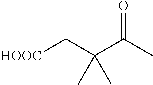 | 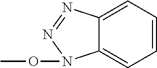 | 0.035 | 2.5 |
| 16 | 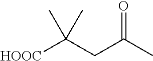 | —NH—(CH₂)₅—NH—COCH₃ | 0.012 | 9 |
| 17 | 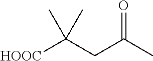 | —NH—(CH₂)₆—NH—COCH₃ | 0.007 | 7.5 |
| 18 (A12-2) | 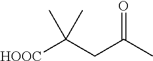 | —NH—(CH₂)₇—NH—COCH₃ | 0.0026 | 8 |
| 19 | 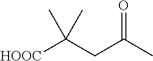 | —NH—(CH₂)₈—NH—COCH₃ | 0.0036 | 7 |
| 20 | 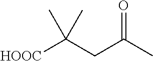 | —NH—(CH₂)₉—NH—COCH₃ | 0.012 | 7.4 |
| 18b (A43-D) | H | —NH—(CH₂)₇—NH—COCH₃ | 0.047 | >10 |

^{a}EC₅₀ is the concentration that inhibits HIV-1 replication by 50%;
IC₅₀ is the drug concentration that resulted in a 50% reduction of viable cells in a 4-day assay.
Data represent an average of at least two experiments.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A compound according to Formula (I): 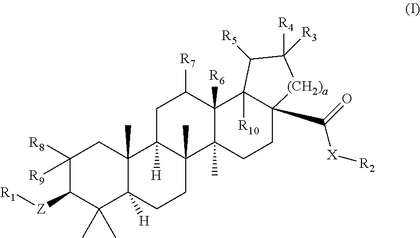
wherein:
a is 1 or 2;
Z is O, S, NH, or N-alkyl;
R₁ is a hydrogen or acyl carboxylic acid,
or R₁ is a substituent of the formula 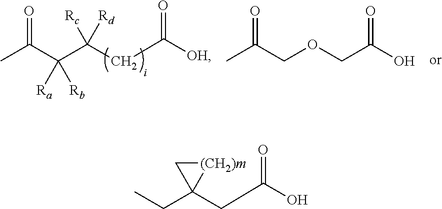
wherein Rₐ, R_{b}, R_{c} and R_{d} are the same or different and are each independently selected from the group consisting of hydrogen and lower alkyl, and i is an integer from 0 to 3, and m is an integer from 1 to 4;
X is O, S, NH, or N-alkyl;
R₂ is a substituent of the formula:
wherein: 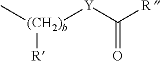
R′ and R″ are either hydrogen or alkyl radicals;
Y is O, S, NH, N-alkyl, or heterocycle;
b is an integer from 0 to 16;
or R₂ is benzotriazole;
R₃ and R₄ are either H or lower alkyl;
R₅ is H, lower alkyl or —CRᵢRᵢᵢRᵢᵢᵢ,
Rᵢ is a methyl radical or forms with Rᵢᵢᵢ a methylene radical or an oxo radical,
Rᵢᵢ is a hydroxyl, methyl or hydroxymethyl radical or a radical —CH₂ OR′ᵢᵢ, —CH₂SR′ᵢᵢ or —CH₂NHR′ᵢᵢ for which R′ᵢᵢ is alkyl, hydroxyalkyl, dihydroxyalkyl, acetamidoalkyl or acetyl, or Rᵢᵢ is an amino radical substituted with a hydroxyalkyl or carboxyhydroxyalkyl radical, or a dialkylamino radical, the alkyl parts of which can fotm, with the nitrogen atom to which they are joined, a 5- or 6-membered heterocycle optionally containing another hetero atom chosen from oxygen, sulphur or nitrogen and, optionally, N-alkyl;
Rᵢᵢᵢ is a hydrogen atom or forms, with Rᵢ or Rᵢᵢ, a methylene radical or an oxo radical,
or R₅ form a bond with its immediately adjacent carbon atoms;
R₆, R₇ are the same or different and are either H or form bond with one another (thus forming a double bond between their immediately adjacent carbon atoms);
R₈, R₉ are the same or different and are either hydrogen or together form an oxo radical;
R₁₀ is either H or form bond with one another (thus faulting a double bond between its immediately adjacent carbon atoms);
or a pharmaceutically acceptable salt or prodrug thereof.

2. A compound of claim 1, wherein R₂ is a substituent of the formula: 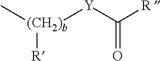
wherein:
R′ and R″ are either hydrogen or alkyl radicals,
Y is O, S, NH, N-alkyl, or heterocycle,
b is an integer from 0 to 16.

3. A compound of claim 1, wherein R₁ is a hydrogen,
or a substituent of the formula 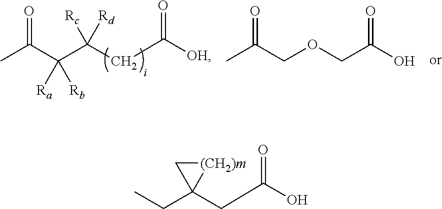
wherein Rₐ, R_{b}, R_{c} and R_{d} are the same or different and are each independently selected from the group consisting of hydrogen or lower alkyl, i is an integer from 0 to 3, and m is an integer from 1 to 4.

4. The compound of claim 1, wherein R₆ and R¹⁰ are each H.

5. The compound of claim 1, wherein R₁ is 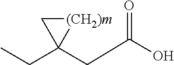
and m is an integer from 1 to 4.

6. The compound of claim 1, wherein R₅ is —CRᵢRᵢᵢRᵢᵢᵢ.

7. The compound of claim 1, wherein R₈ and R₉ are each H.

8. The compound of claim 1, wherein Rᵢ and Rᵢᵢᵢ together form a methylene radical.

9. The compound of claim 1, wherein Rᵢᵢ is methyl.

10. The compound of claim 1, wherein R₃ and R₄ are each H.

11. The compound of claim 1, wherein R₆ and R₇ are each H.

12. The compound of claim 1 having the following formula: 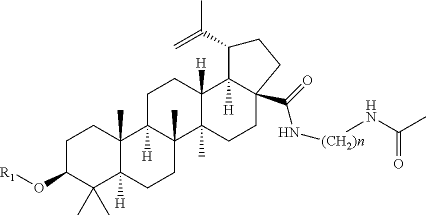
wherein R₁ is either H or 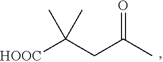
and n is an integer from 1-10, or a pharmaceutically acceptable salt or prodrug thereof.

13. The compound of claim 1 selected from the group consisting of 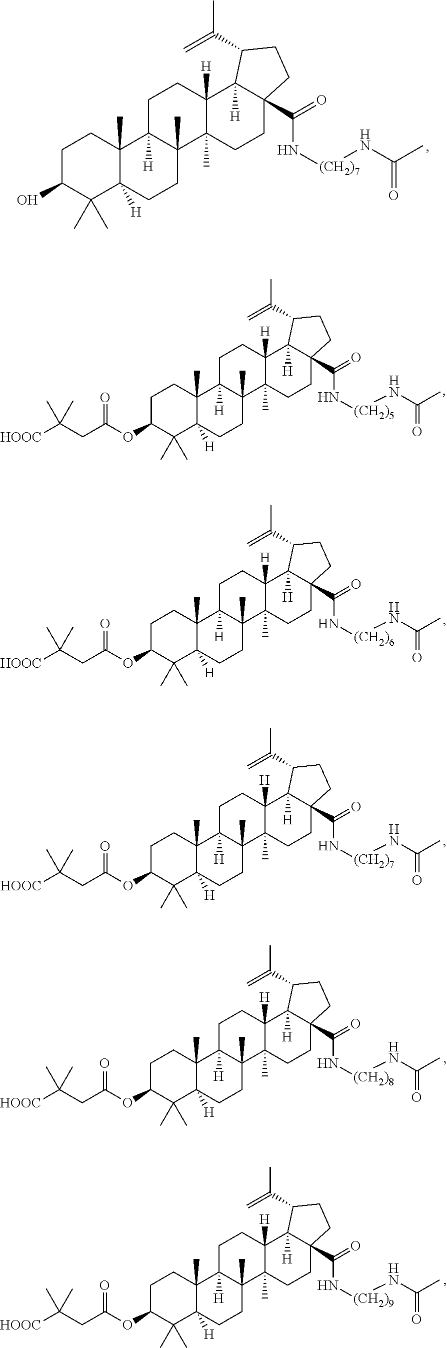
and pharmaceutically acceptable salts or prodrugs thereof.

14. A composition comprising a compound of claim 1 in a pharmaceutically acceptable carrier.

15. The composition according to claim 14, wherein said carrier comprises an aqueous solution.

16. The composition of claim 14, further comprising an HIV entry inhibitor.

17. A method of treating a viral infection in a subject in need thereof, comprising administering to said subject a compound of claim 1 in an amount effective to treat said viral infection.

18. The method of claim 17, wherein said viral infection is an HIV-1 infection.

19. The method of claim 17 wherein said HIV-1 infection is a DSB-resistant HIV-1 infection.

20. The method of claim 17, wherein said HIV-1 infection is an RPR103611-resistant HIV-1 infection.

21. The method of claim 17, further comprising concurrently administering to said subject another HIV entry inhibitor.
